(19) United States
(12) Patent Application Publication
(10) Pub. No.: US 2018/0103817 A1
(43) Pub. Date: Apr. 19, 2018

(54) CARPET CLEANER

(71) Applicant: QUENTIN NADAL, Indianapolis, IN (US)

(72) Inventor: Quentin Nadal, Indianapolis, IN (US)

(21) Appl. No.: 15/786,936

(22) Filed: Oct. 18, 2017

Related U.S. Application Data
(60) Provisional application No. 62/410,059, filed on Oct. 19, 2016.

(51) Int. Cl. A47L 11/40; A61L 2/26

(52) U.S. Cl. CPC A47L 11/4044; A47L 11/4094; A47L 11/4013; A47L 11/34; A61L 2/26; A61L 2202/11; A61L 2202/17; A47L 11/4088

(57) ABSTRACT

Certain embodiments of the present disclosure describe a surface cleaning system, particularly for use in cleaning carpets. The surface cleaning system includes an external tank, a connection line, and a wand assembly. The wand assembly is detachably connected to the connection line at a handle portion. A vacuum motor is included on the wand assembly to provide suction when the wand assembly is detached from the connection line. A wand head included on the wand assembly includes a suction tube attached to the vacuum motor and an internal wand head cavity that may hold a brush. An internal tank adjacent the internal wand head cavity may hold liquid and debris that is picked up by the suction supplied to the suction tube and/or the wand head cavity. Liquid from the internal tank may be pumped or suctioned to the external tanks and discharged through an outlet line.

### CROSS REFERENCE TO RELATED APPLICATIONS

The present application claims the benefit of U.S. Provisional Patent Application, Ser. No. 62/410,059 filed Oct. 19, 2016, which is hereby incorporated by reference.

### BACKGROUND

This disclosure is in the field of surface cleaners, particularly in the field of carpet cleaners.

Hot water extraction or steam cleaning is a commonly used method for cleaning a surface such as carpet. This method deposits hot water onto a surface and then a cleaning tool is passed over the surface to remove the water, often through the use of suction. Machines such as a water pump and vacuum motors that are used in the steam cleaning process are often large and may be mounted to trucks. This may be suitable for some cleaning applications, but some cleaning jobs may require more portable tools that are able to get to hard to reach places. Therefore, it may be desirable for a surface cleaning system to include features that allow for greater portability.

### BRIEF DESCRIPTION OF THE DRAWINGS

FIG. 1 is a schematic diagram of a surface cleaner.

FIG. 2 is a schematic diagram of an external tank of the surface cleaner of FIG. 1.

FIG. 3 is a diagram of a wand assembly of the surface cleaner of FIG. 1.

FIG. 4 is a diagram of a solution reservoir of the surface cleaner of FIG. 1.

FIG. 5 is a diagram of a cleaning system of the surface cleaner of FIG. 1.

FIG. 6 is a cross-sectional view of an embodiment of a wand head usable with the surface cleaner of FIG. 1.

FIG. 7 is a cross-sectional view of another embodiment of a wand head usable with the surface cleaner of FIG. 1.

FIG. 8 is a cross-sectional view of yet another embodiment of a wand head usable with the surface cleaner of FIG. 1.

FIG. 9 is an internal view of an embodiment of a wand head for the surface cleaner of FIG. 1.

FIG. 10 is an internal view of an embodiment of a wand head for the surface cleaner of FIG. 1.

FIG. 11 is a schematic diagram of an embodiment of a reserve tank of the surface cleaner of FIG. 1.

FIG. 12 is a cross-sectional view of an alternative embodiment of a connection line of the surface cleaner of FIG. 1.

FIG. 13 is a schematic diagram of an alternative embodiment of an external tank usable with the surface cleaner of FIG. 1.

FIG. 14 is a schematic diagram of an alternative embodiment of a surface cleaner.

FIG. 15 is a schematic diagram of another alternative embodiment of a surface cleaner.

### DETAILED DESCRIPTION OF THE DRAWINGS

Reference will now be made to certain embodiments and specific language will be used to describe the same. It will nevertheless be understood that no limitation of the scope of this disclosure and the claims is thereby intended, such alterations, further modifications and further applications of the principles described herein being contemplated as would normally occur to one skilled in the art to which this disclosure relates. In several figures, where there are the same or similar elements, those elements are designated with the same or similar reference numerals.

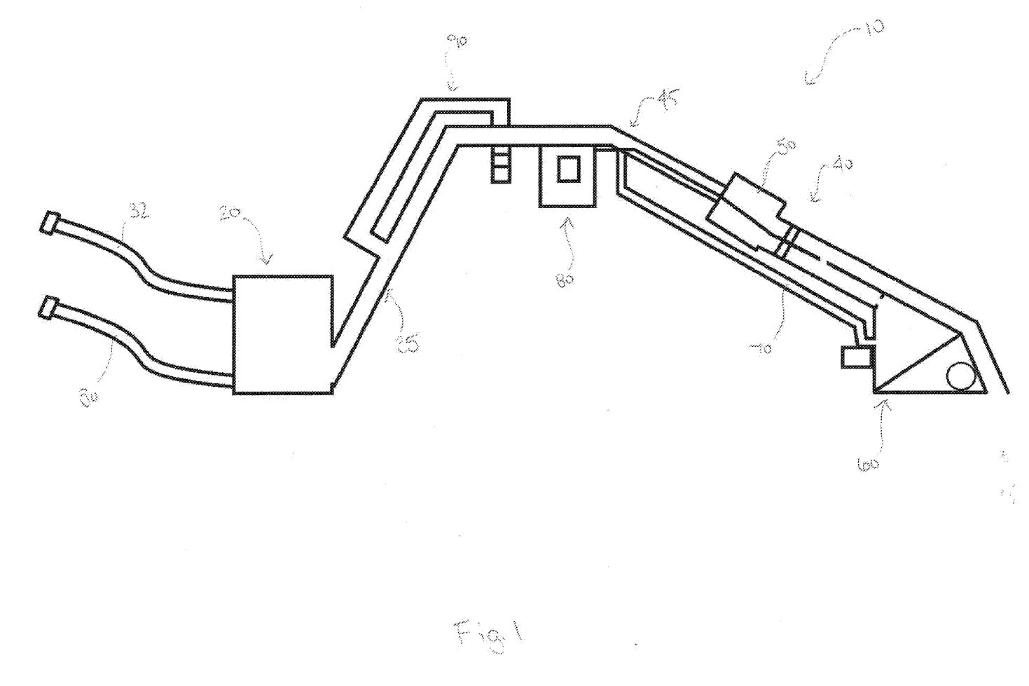

The present disclosure pertains to a surface cleaner that may be operated for either steam cleaning or for dry cleaning. FIG. 1 is a schematic view of a surface cleaning system 10. Surface cleaning system 10 includes an external tank 20, a connection line 35 that connects external tank 20 to a handle portion 45 of a detachable wand assembly 40. The other end of wand assembly 40 includes a wand head 60. Handle portion 45 of wand assembly 40 may optionally include a solution reservoir 80. Additionally, an optional cleaning system 90 may be included in connection line 35.

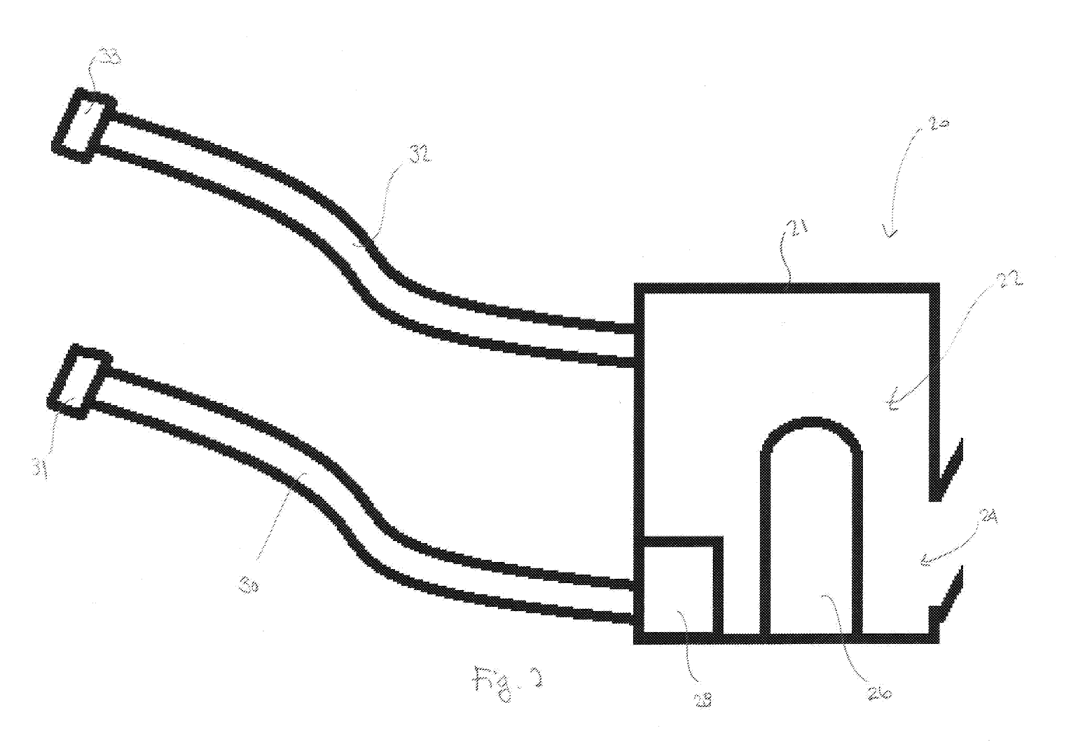

As seen in FIG. 2, external tank 20 includes a housing 21 that defines an internal area 22. Internal area 22 may be used as a reservoir to hold excess liquid that has been suctioned from surface cleaning system 10. An opening 24 in housing 21 may be used to connect external tank 20 to connection line 35, which fluidly connects external tank 20 to wand assembly 40.

Internal area 22 may also include a vacuum motor 26 that may provide a source of suction to surface cleaning system 10 through connection line 35 to wand head 60. A pump 28 may also be positioned in internal area 22. Pump 28 may remove the excess liquid that is held in internal area 22 by pumping the water through a discharge line 30. Discharge line 30 may be a hose or any other structure suitable for allowing a liquid to flow through. An end 31 of discharge line 30 that is not attached to external tank 20 may be positioned at a sink or a drain so that the excess liquid removed from internal area 22 is pumped into a suitable removal area.

An input line 32 may also be attached to external tank 20. An external end 33 of input line 32 may be attached to a water source such as a sink or a water spigot to provide a pressurized source of fresh water. As described below, the fresh water from input line 32 may travel through connection line 35 to wand assembly 40. In some embodiments, input line 32 may bypass external tank 20 and be attached directly to connection line 35 rather than being attached to external tank 20.

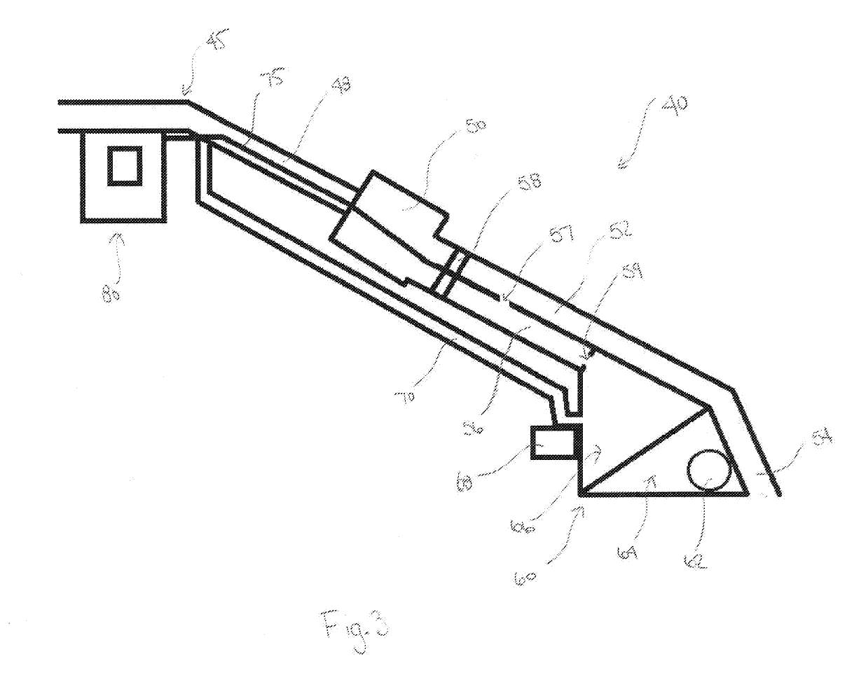

FIG. 3 shows wand assembly 40 of surface cleaning system 10. Handle portion 45 of wand assembly 40 is detachably connected to connection line 35 so that wand assembly 40 may be used in conjunction with external tank 20, or wand assembly 40 may be used on its own. A tube 48 extends between handle portion 45 and one end of a vacuum motor 50. Motor 50 may be used to create a vacuum that provides wand assembly 40 with suction independent of any suction that is provided by vacuum motor 26 in external tank 22. This allows wand potion 40 to be operated even while detached from external tank 22, so that surface cleaning system 10 may be more portable.

A pair of tubes 52, 56 extend from vacuum motor 50 with filter 58 positioned between vacuum motor 50 and suction tube 52. Suction tube 52 extends from motor 50 and around wand head 60 to an end portion 54 of suction tube 52 that may be positioned on a floor or other surface that is to be cleaned. Suction created by motor 50 allows liquid, dirt, or other solids to be sucked through end portion 54 of suction tube 52.

Wand head tube 56 extends parallel to a portion of suction tube 52 between motor 50 and wand head 60. Wand head tube 56 is in fluid connection with suction tube 52 due to an aperture 57 and is in fluid connection with wand head 60 through a second aperture 59.

Wand head 60 includes a brush 62 positioned in a wand head cavity 64. An internal tank 66 is positioned adjacent to wand head cavity 64. A pump 68 may be positioned within or adjacent to internal tank 66 and may be used to pump excess liquid held in internal tank 66 through a return line 70 which sends the excess liquid to the external tank 20. In some embodiments, return line 70 may be connected to wand assembly 40 at handle portion 45. The excess liquid is pumped through return line 70 and then through connection line 35 to external tank 20 or directly to a discharge line.

In some embodiments, instead of using a pump 68 to remove liquid from internal tank 66, suction may be used to remove liquid from internal tank 66. As an example, return line may be fluidly connected to external tank 20 so that suction provided by vacuum motor 26 may be used to remove liquid from internal tank 66. This can provide a “double vacuum” configuration where both vacuum motor 50 and vacuum motor 26 provide suction at end portion 54.

In use, surface cleaning system 10 may be used either for steam cleaning or for dry cleaning. If used for steam cleaning, a cleaning liquid such as water is supplied through an input line 32. As an example, input line 32 may be connected to a water supply such as a sink or a water spigot. The liquid supplied through input line 32 may be pumped through connection line 35, to the handle portion 45 of wand assembly 40, and then through wand head 60 to the surface to be cleaned. Suction provided through suction tube 52 may then be used to remove the liquid and any other dirt from the surface.

Suction for the wand assembly 40 is created by vacuum motor 50. In some embodiments, additional suction may be provided by vacuum motor 26 located in external tank 20. Motor 50 allows wand assembly 40 to operate even if wand assembly 40 is detached from connection line 35. The detachable wand assembly 40 may provide for greater mobility and allow easier access to places that may be difficult to clean if wand assembly 40 is attached to connection portion 50 and an external tank 20.

The suction created by vacuum motor 50, and in some embodiments also by vacuum motor 26, may be provided to end portion 54 of suction tube 52 and may also be provided to wand head cavity 64. As described below, wand head 60 may optionally include a valve that allows a user to choose where suction is provided. For example, in some embodiments, the valve may be set so that suction is only provided to end portion 54 of suction tube 52 or so that suction is only provided to wand head cavity 64. In other embodiments, the valve may be set so that suction is provided to both end portion 54 and to wand head cavity 64.

As liquid is suctioned through end portion 54, the liquid travels through suction tube 52 toward motor 50. The liquid travels to aperture 57 where the liquid exits suction tube 52 and enters wand head tube 56. Then, either through gravity or a pump, the liquid travels along wand head tube 56 to an aperture 59 where it enters internal tank 66. Likewise, if suction is provided to wand head cavity 64, any liquid or debris that is suctioned through wand head cavity 64 may pass through wand head cavity 64 and into internal tank 66.

When the wand assembly 40 is detached from external tank 20, any liquid picked up by wand assembly 40 remains in internal tank 66 until it is manually emptied by the user. When wand assembly 40 is attached to external tank 20, the liquid may be pumped or suctioned through return line 70. The liquid travels through return line 70 to handle portion 45, then through connection line 35 where it is delivered to external tank 20. The liquid then may be held in external tank 20, or may be pumped out of external tank 20 through discharge line 30.

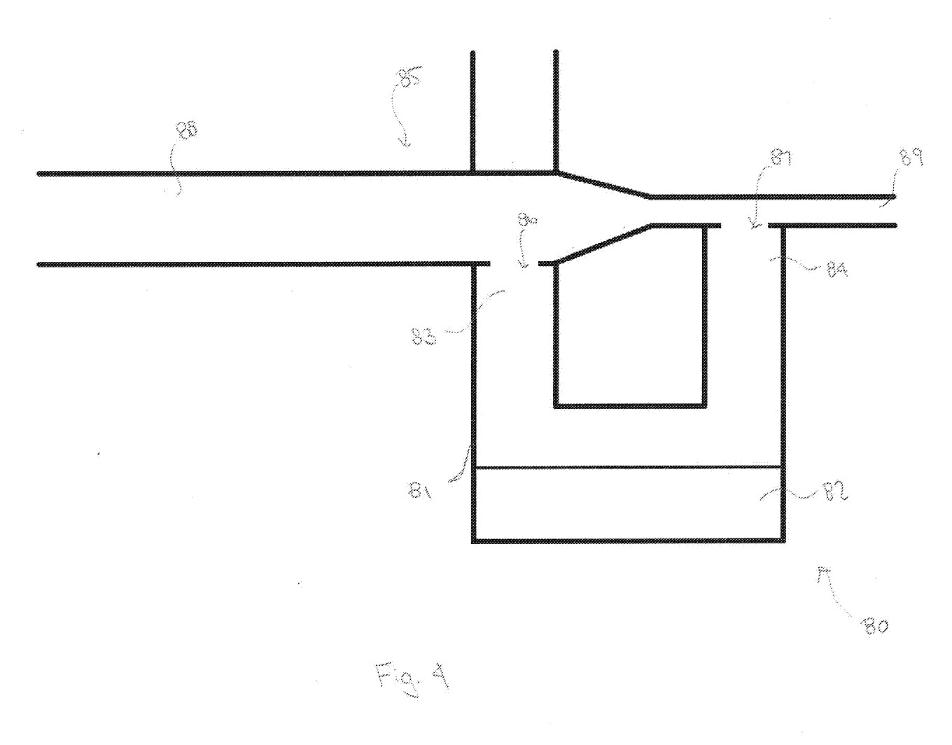

Optionally, a solution reservoir 80 may be attached to handle portion 45. As shown in FIG. 4, solution reservoir 80 includes a solution tank 81 that holds a cleaning solution 82. Solution reservoir 80 is in fluid communication with a liquid supply line 85, so that a cleaning liquid, such as water, flowing through liquid supply line 75 may be mixed with the cleaning solution 82 held in solution tank 81. In some embodiments, liquid supply line 85 may be connection line 35, so that solution reservoir 80 is directly in line with connection line 35. In other embodiments, liquid supply line 85 may be separate from and attached to connection line 35. In those embodiments, a portion of the liquid from connection line 35 may be introduced into liquid supply line 85.

In the embodiment shown, solution tank 81 has an entrance portion 83 and an exit portion 84, so that solution tank 81 is U-shaped. An entrance orifice 86 connects the liquid supply line 85 to entrance portion 83. An exit orifice 87 connects exit portion 84 to liquid supply 85 at a position further downstream than entrance orifice 86. An upstream portion 88 of liquid supply line 85 has a larger diameter than downstream portion 89 of liquid supply line 85.

In the embodiment of solution reservoir 80 shown in FIG. 4, the Venturi effect is used to mix solution 82 with a liquid flowing through liquid supply line 85. The reduction of diameter in liquid supply line 85 between upstream portion 88 and downstream portion 89 creates a pressure differential between entrance portion 83 of solution tank 81 and exit portion 84 of solution tank 81. Due to the increased velocity of the liquid in downstream portion 89 compared to upstream portion 88 because of the reduced diameter of liquid supply line 85, the pressure at exit portion 84 of solution tank 81 is less than the pressure at entrance portion 83. The decreased pressure on exit portion 84 pushes solution 82 through exit orifice 87, where it is mixed with the liquid in liquid supply line 85.

Downstream portion 89 of liquid supply line 85 may be attached to a solution line 75 that is in fluid communication with wand head 60 so that the solution may be introduced into wand head cavity 64. The liquid and solution mixture may be pumped through solution line 75 to wand head cavity 64, where it is released onto the surface that is to be cleaned.

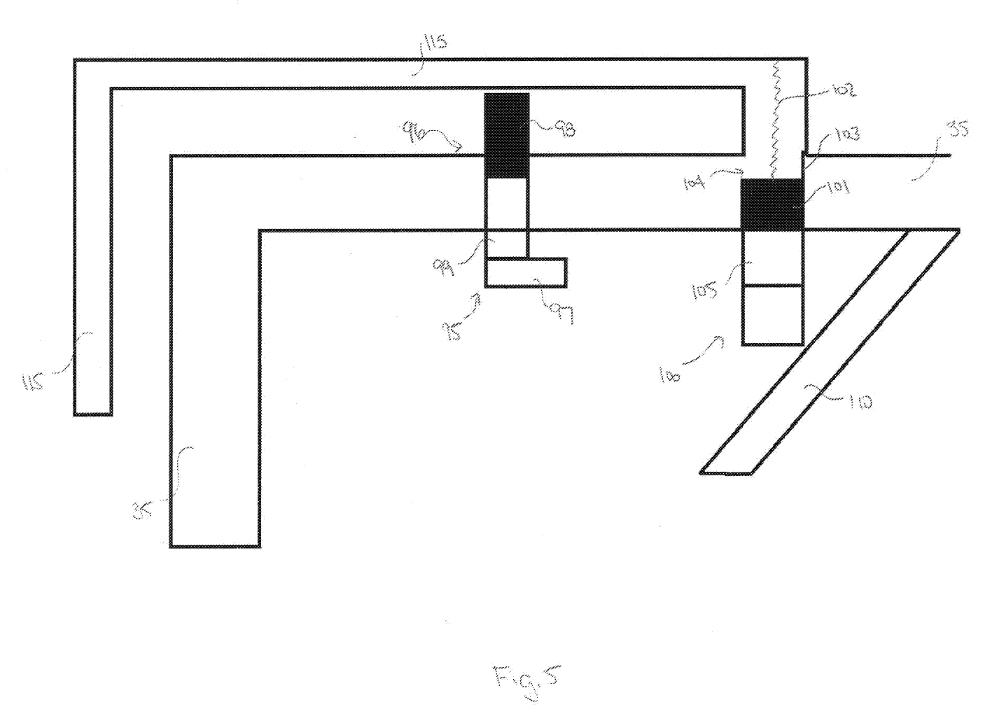

FIG. 5 shows an embodiment of a cleaning system 90. In some embodiments, cleaning system 90 includes a valve portion 95 that is positioned on connection line 35. Valve portion 95 includes a slidable member 96 and a toggle 97. Slidable member 96 includes a blocker 98 that prohibits the flow of fluid through connection line 35 when blocker 98 is positioned within connection line 35. Another portion of slidable member 96 defines an opening 99 that permits the passage of liquid when positioned within connection tube 99. The position of slidable member 96 may be adjusted by either pushing or pulling on toggle 97.

A return valve 100 is positioned downstream of valve 95. Return valve 100 includes a plug 101 that at least partially prevents the passage of liquid when positioned within connection line 35. When return valve 100 is in a closed position, plug 101 may partially close connection line 35, while an extension 103 covers the rest of connection line 35 to prevent the flow of liquid further through connection line 35. Because plug 101 is smaller than connection line 35, an opening 104 is defined between plug 101 and connection line 35. This opening 104 allows liquid to flow from connection line 35 into a return tube 115.

An orifice 105 defined in return valve 100 may be positioned within connection line 35 to allow the passage of liquid. A lever 110 may be used to allow position valve 100 in a closed position where plug 101 is positioned within connection line 35 and an open position where orifice 105 is positioned within connection line 35. In this position, plug 101 covers return tube 115 so that no liquid is supplied to return tube 115. In other embodiments, lever 110 may not be necessary, and any other suitable means of moving return valve may be used.

There are three possible states for return valve 100. In a first state, all of the liquid in connection line 35 is directed to return tube 115. A resilient member 102 connected to return valve 100 at plug 101 biases return valve 100 to be positioned in the closed position where all of the liquid in connection line 35 is diverted through return tube 115 where it may be returned to external tank 22 or otherwise disposed.

A user may push upward on lever 115 to overcome the biasing force of resilient member 102, and place return valve 100 in a second state where some of the liquid from connection line 35 is allowed to pass through valve 100 and where a portion of the liquid is diverted to return tube 115. In this state, lever 115 is used to raise plug 101 so that a portion of plug 101 covers connection line 35 and a portion of orifice 105 is positioned within connection line 35 to allow the passage of liquid. Plug 101 is positioned so that it does not entirely occlude return tube 115.

In a third state, return valve 100 is placed in an open position by pushing further upward on lever 115 so that orifice 105 is fully positioned within connection line 35 and plug 101 is positioned to fully cover the entrance to return tube 115. When orifice 105 is positioned within connection line 35, all of the liquid is allowed to flow through connection line 35 toward wand assembly 40, and none of the liquid is diverted to return tube 115.

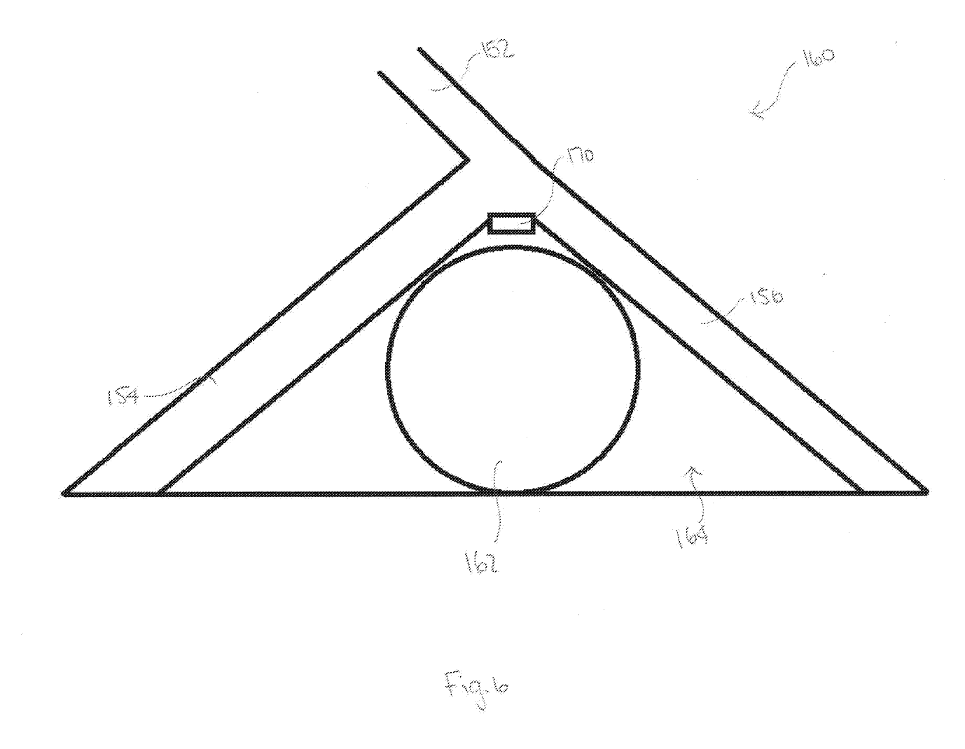

Different embodiments of wand heads may be used in alternative embodiments of surface cleaning system 10. FIG. 6 shows an embodiment of a wand head 160. Wand head 160 includes a suction tube 152 that branches into two sections 154, 156 that surround a wand head cavity 164. A brush 162 may be positioned within wand head cavity 164. Suction may be introduced through suction cavity 152 so that liquid may be suctioned through sections 154, 156.

Wand head 160 also includes a valve 170 that may be either open or closed. When valve 170 is closed, no suction is provided to wand head cavity 164. However, when valve 170 is opened, suction is provided to wand head cavity 164 so that dirt and other objects picked up through wand head cavity 164 may be removed from the surface being cleaned.

Valve 170 allows wand head 160 to be used for both dry and steam cleaning. When only steam cleaning is desired, valve 170 may be closed so that section 154, 156 are used for steam cleaning, but dry cleaning using wand head cavity 164 is not performed. When dry cleaning is desired, valve 170 is opened so that suction comes from sections 154, 156 and also from wand head cavity 164.

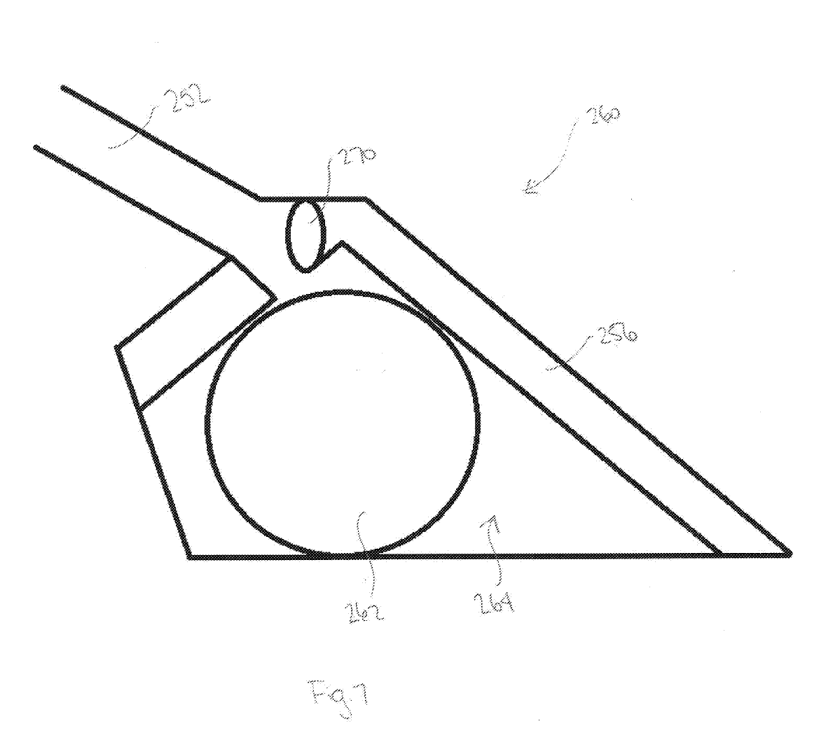

FIG. 7 shows an embodiment of a wand head 260 that includes a valve 270 that may be positioned to provide for either steam cleaning or dry cleaning. Wand head 260 is attached to a suction tube 252. A section 256 of suction tube 252 extends around a wand head cavity 264. A brush 262 is positioned within wand head cavity 264.

In FIG. 7, valve 270 within wand head 260 is positioned to block suction from section 256 of suction tube 252 so that wand head 260 may be used for dry cleaning. Suction from suction tube 252 acts to pull up dirt and other objects picked up by brush 262. Valve 270 may alternatively be positioned so that it blocks access to wand head cavity 264 and so that suction is provided to section 256 of suction tube 252. This arrangement may be desired for steam cleaning where the smaller suction area provided by section 256 may be desired rather than suction area provided by wand head cavity 264.

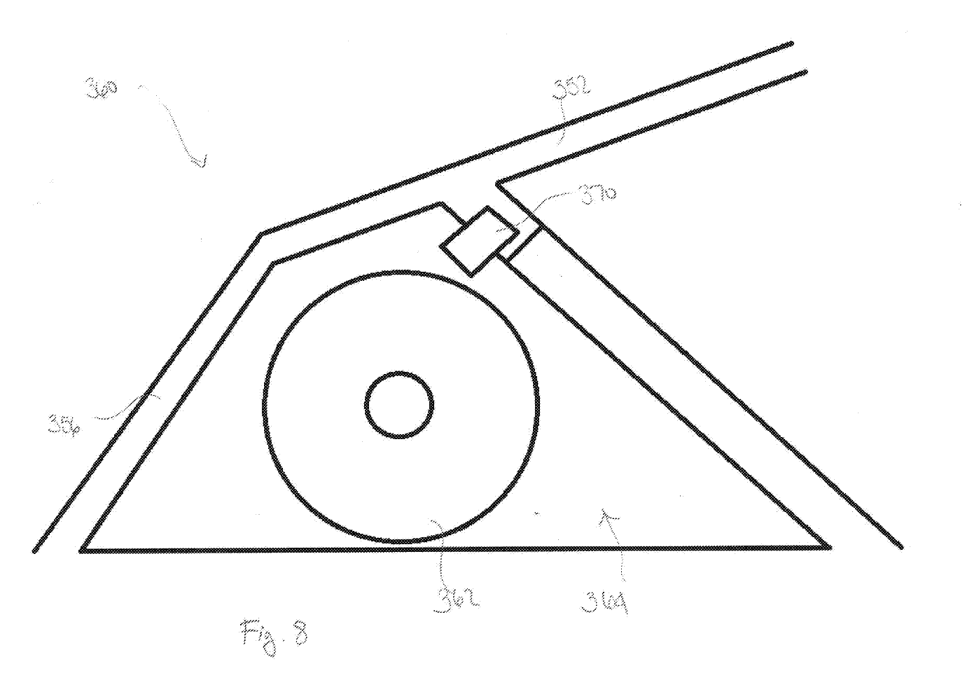

FIG. 8 shows an embodiment of a wand head 360 that allows for suction through a small opening or optionally through both a small opening and a large opening. Wand head 360 is connected to a suction tube 352. A portion 356 of suction tube 352 is adjacent a wand head cavity 364. A brush 362 may be positioned within wand head cavity 364. A valve 370 may be selectively opened are closed to either allow or to disallow suction to wand head cavity 364.

For steam cleaning, when a smaller suction area is desired, valve 370 may be closed so that suction is only provided through section 356 of suction tube 352. For dry cleaning, when a larger suction area may be desired, valve 370 is opened so that suction is provided to both section 356 and to wand head cavity 364.

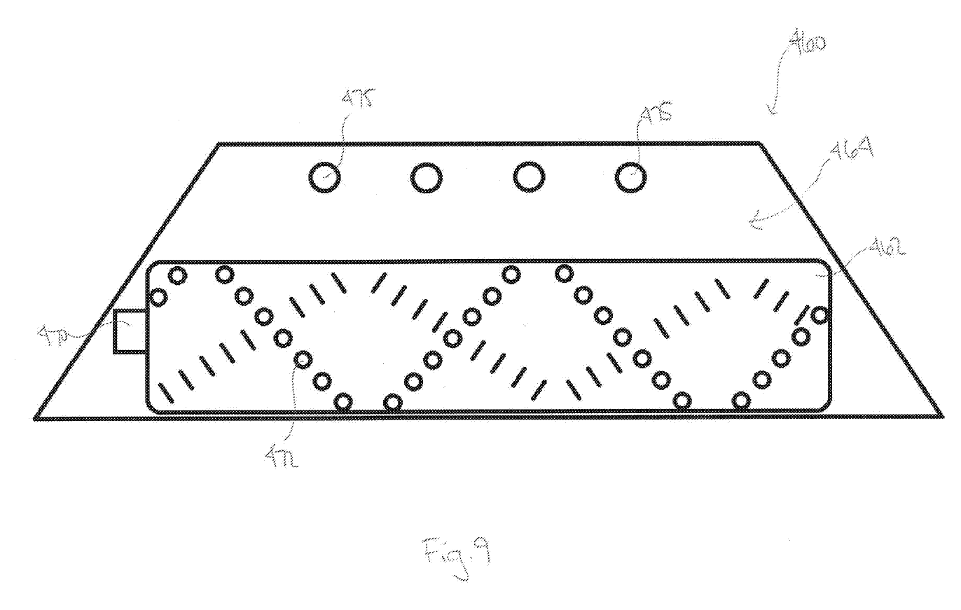

An embodiment of a self-cleaning wand head 460 is shown in FIG. 9. Wand head 460 includes a wand head cavity 464 and a brush 462 positioned within wand head cavity 464. Brush 462 includes a liquid inlet 470 for receiving a cleaning liquid such as a cleaning solution or water. Brush 462 also includes holes 472 along the outer surface of brush 462 that are in fluid connection with liquid inlet 470. Liquid that is pumped through liquid inlet 470 is pushed out of holes 472 to clean the interior of wand head cavity 464 and to provide liquid to the surface beneath brush 462.

In the embodiments shown, holes 472 are arranged diagonally and around the circumference of brush 462. Other embodiments may include any other desired arrangement of holes 472. For example, holes 472 may be positioned in multiple rings wrapped circumferentially around brush 462. In other embodiments holes may be randomly distributed on brush 462.

In some embodiments wand head 460 may also include additional openings 475 within wand head cavity 464. Water or any other desired cleaning liquid may be introduced through openings 475 and sprayed within wand head cavity 464. As an example, water supplied from input line 32 may be pumped through external tank 20, connection line 35 and wand assembly 40 to opening 475.

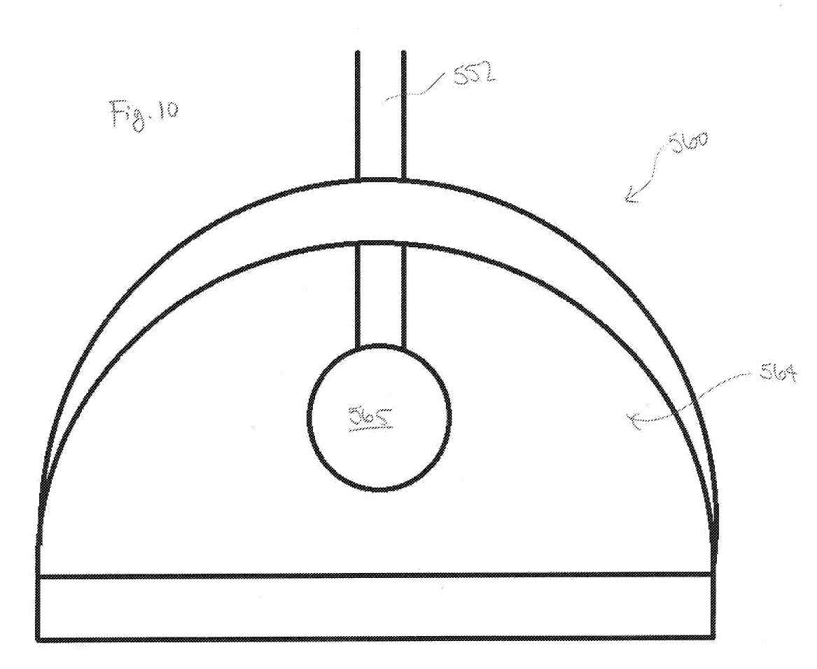

An embodiment of a wand head 560 without a brush is shown in FIG. 10. In this embodiment, wand head 560 is connected to a suction tube 552 that ends at a suction opening 565 within a wand head cavity 564 of wand head 560. Suction from a vacuum motor is provided through suction tube 552. Dirt and other objects passed over by wand head cavity 564 may be picked up by the suction and directed into opening 565. The dirt then passes through suction tube 552 and is directed to an internal or external tank.

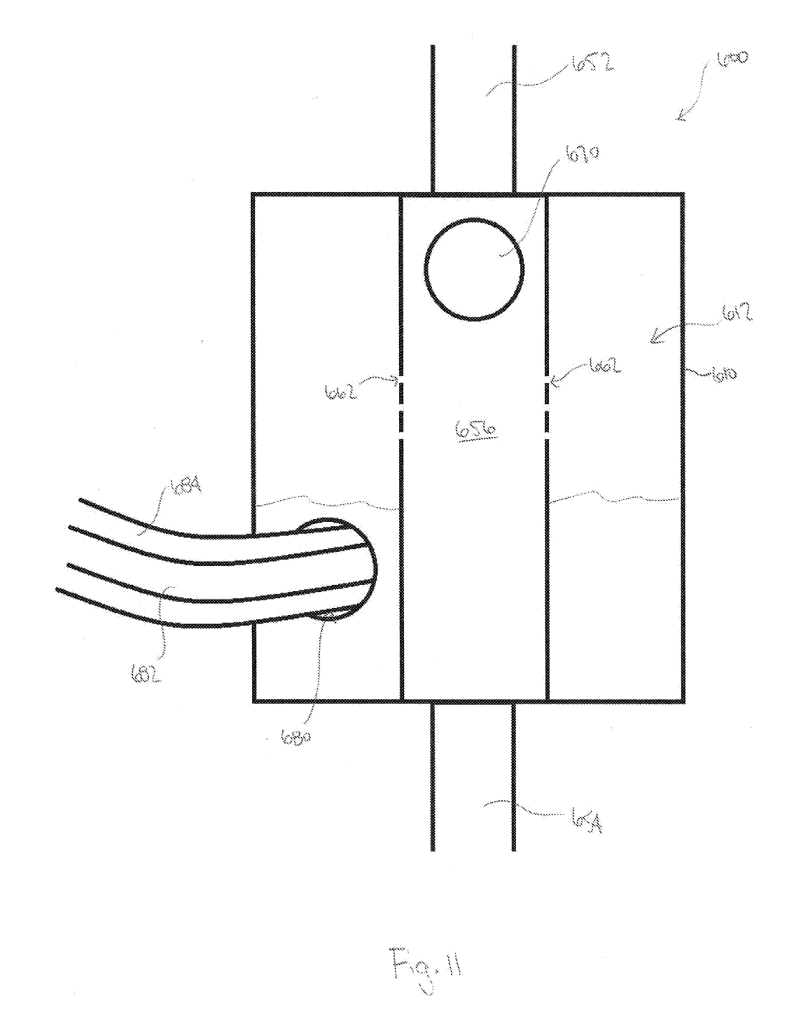

FIG. 11 shows an embodiment of a reserve tank 600 that may be used to hold water that has been suctioned through cleaner 10. Reserve tank 600 includes a housing 610 that defines an internal cavity 612. A tank tube 656 extends through internal cavity 612. One end of tank tube 656 is a connected to a tube 652 that extends along the wand toward a vacuum motor (not shown). The other portion of tank tube 656 is connected to a tube 654 that extends to a wand head 60. Apertures 662 defined in tank tube 656 allow liquids and other small particles to exit tank tube 656 and to be held within internal cavity 612. A filter 670 in tank tube 656 prevents large objects from entering into tube 652 and entering the vacuum motor.

A sealed opening 680 in tank housing 610 provides access for a pair of hoses 682, 684 into internal cavity 612. In some embodiments, hoses 682 and 684 may be arranged concentrically so there is an inner hose 682 and an outer hose 684. Liquid held in internal cavity 612 may pumped through internal hose 682 toward an external source, such as external tank 20, to remove the liquid from internal cavity 612. In some embodiments, a vacuum source may provide suction to remove liquid from internal cavity 612. In some embodiments, the concentric outer hose 684 may be connected to another vacuum source to provide additional suction to aid in removing liquid from internal cavity 612.

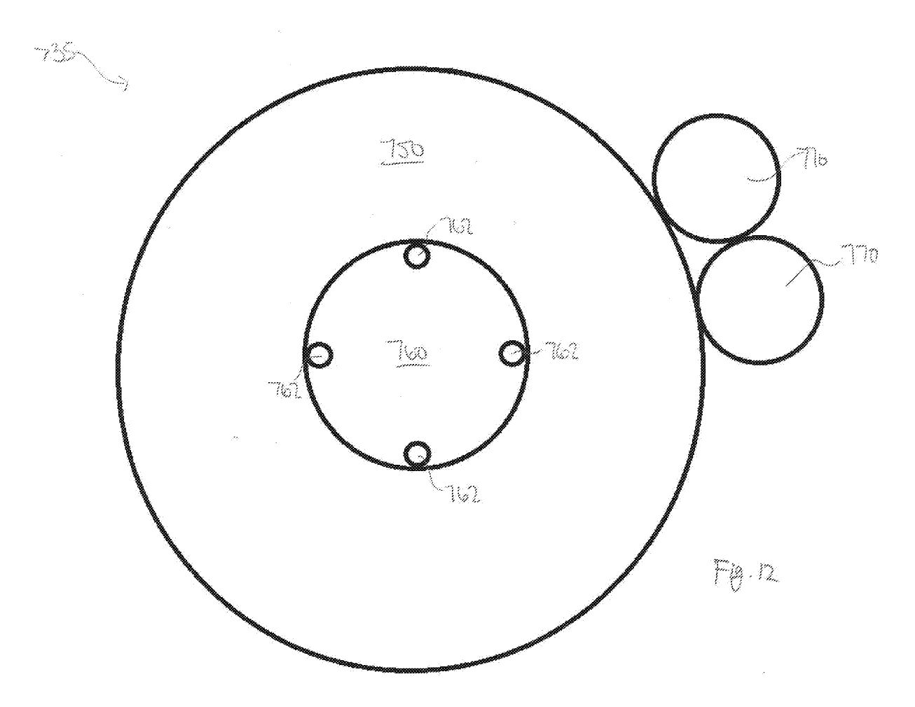

A cross-sectional view of an embodiment of a connection line 735 of a surface cleaning system 10 is shown in FIG. 12. Connection line 735 includes concentric tubes 750, 760 that extend along connection line 735. Power lines 770 may optionally be positioned on the outside of tube 750. Tube 750 is a suction tube that is operationally connected to a vacuum source. Tube 760 is a water tube that is operationally connected to a water source that provides a supply of fresh water such as from tube 115. Water may be sprayed into tube 750 through apertures 762 in water tube 760. This water may be sprayed into tube 750 to clean tube 750 including while tube 750 is used to suction and transport dirty water between a wand assembly and an external tank.

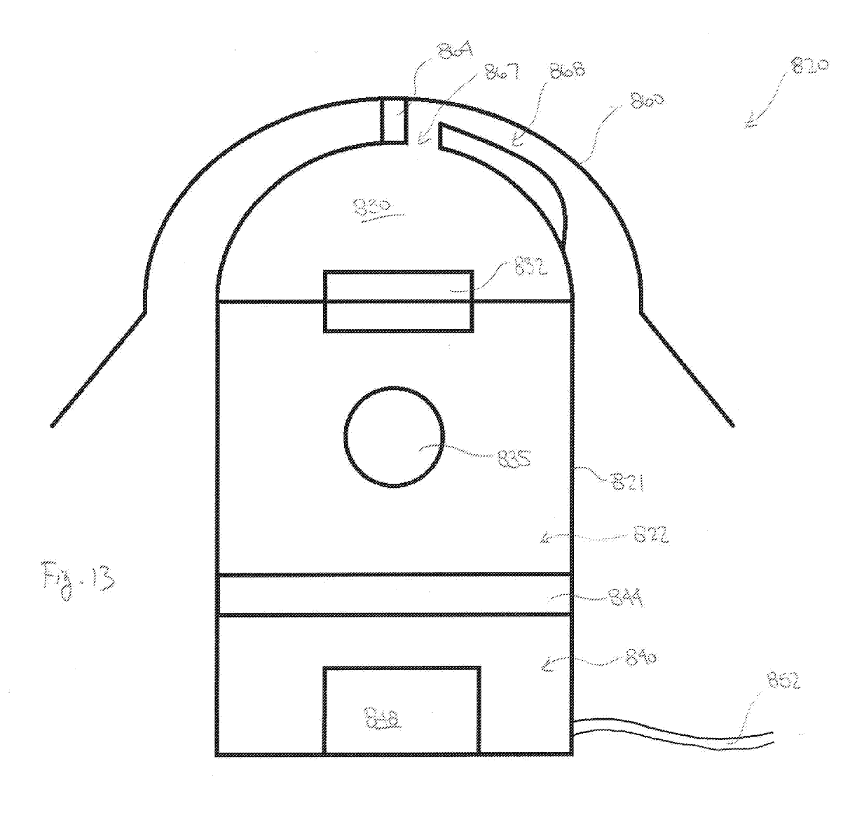

FIG. 13 illustrates an alternative embodiment of an external tank 820. External tank 820 includes a housing 821 that defines an internal area 822. Internal area 822 includes a vacuum motor 830 for supplying suction to cleaner 10 and a filter 832 for the vacuum motor. An inlet 835 provides an opening for connecting connection line 35.

The bottom portion of internal area 822 of housing 821 includes a reservoir 840 for holding excess liquid that is suctioned by vacuum motor 830. A filter 844 prevents solids that are also suctioned by vacuum motor 830 from entering reservoir 840. A pump 848 may be positioned within reservoir 840 to pump a liquid within reservoir 840 through an outlet line 852 to an external area such as a drain.

External tank 820 includes a rotatable lid 860 that is attached to the top of housing 821 and above vacuum motor 830 at a rotation point 864. Rotation point 864 may be any structure that allows rotation of lid 860 about housing 821. Exhaust air from vacuum motor 830 is pushed through an opening 867 that leads to lid 860. The air is then directed through path 868 and exits lid 860 at in a downward direction toward the surface on which external tank 820 sits. The exhaust air may be used as a dryer to assist in drying the surface. The movement of exhaust air through path 868 of lid 860 may also cause lid 860 to rotate about rotation point 864 so that the air is exhausted in a circular pattern and is able to continuously rotate and dry the entire surface around external tank 820 by making full rotations around external tank 820.

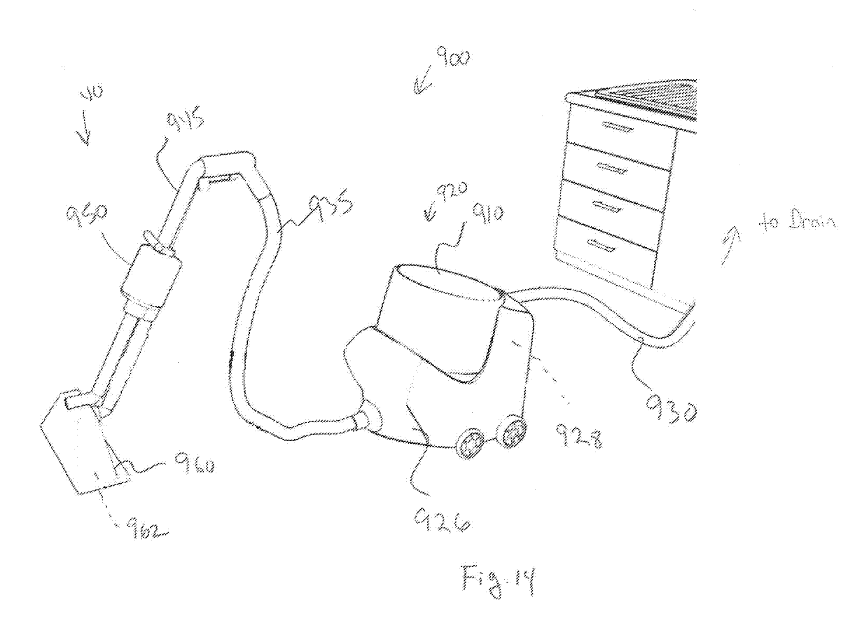

FIG. 14 illustrates surface cleaning system 900. Surface cleaning system 900 generally includes remote assembly 920 and wand assembly 940 connected by connection line 935. Wand assembly 940 includes suction system 950, wand head 960 and handle portion 945. Wand head 960 may also optionally include rotating brush 962 contained within wand head 960 (not illustrated, but could be similar to one of brush 262, 362 or 462 described above).

In the illustrated embodiment, suction system 950 is a blower configured with the inlet fluidly coupled to wand head 960 and the outlet fluidly coupled to connection line 935. In this configuration, everything extracted through wand head 960 including liquids, solids, suspended solids and air pass through suction system 950 and in particular, the fan in suction system 950. Connection line 935 is a combination vacuum and water line such that connection line 935 includes separate passageways for water heading toward wand assembly 940 and a fluid/air passageway for material leaving want assembly.

Remote assembly 920 includes tank 910, vacuum 926 and pump 928. discharge line 930 fluidly connects remote assembly 920 to a repository for waste materials, such as a drain or a vehicle mounted storage tank (not illustrated). In the illustrated embodiment, discharge line 930 includes multiple passageways including one passageway for water heading toward remote assembly 920 and a second passageway for fluid and suspended solids pumped to the drain or vehicle mounted storage tank. Remote assembly 920 is coupled to wand assembly 940 by connection line 935. Vacuum 926 work with suction system 950 to pull fluid and air from wand assembly 940 to tank 910. Pump 928 transfers fluid, and some or all solids suspended in the fluid, through discharge line 930 to the connected drain or vehicle mounted storage tank.

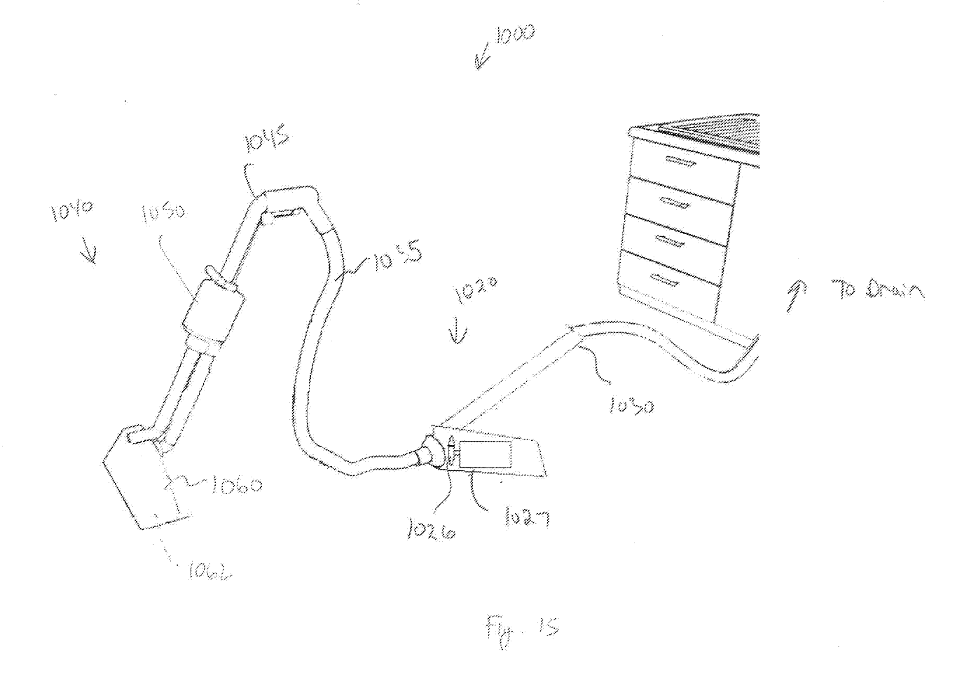

FIG. 15 illustrates surface cleaning system 1000. Surface cleaning system 1000 generally includes remote assembly 1020 and wand assembly 1040 connected by connection line 1035. Wand assembly 1040 includes suction system 1050, wand head 1060 and handle portion 1045. Wand head 1060 may also optionally include rotating brush 1062 contained within wand head 1060 (not illustrated, but could be similar to one of brush 262, 362 or 462 described above).

In the illustrated embodiment, suction system 1050 is a blower configured with the inlet fluidly coupled to wand head 1060 and the outlet fluidly coupled to connection line 1035. In this configuration, everything extracted through wand head 1060 including liquids, solids, suspended solids and air pass through suction system 1050 and in particular, a fan in suction system 1050. Connection line 1035 is a combination vacuum and water line such that connection line 1035 includes separate passageways for water heading toward wand assembly 1040 and a fluid/air passageway for material leaving want assembly.

Remote assembly 1020 includes fan 1026 driven by motor 1027. The inlet to fan 1026 is coupled to connection line 935 and the outlet from fan 1026 is connected to discharge line 1030. In this configuration, all fluids, solids and air extracted through wand head 960 pass through fan 1026. Discharge line 1030 fluidly connects remote assembly 1020 to a repository for waste materials, such as a drain or a vehicle mounted storage tank (not illustrated). In the illustrated embodiment, discharge line 1030 includes multiple passageways including one passageway for water heading toward remote assembly 1020 and a second passageway for fluid and suspended solids pumped to the drain or vehicle mounted storage tank. Remote assembly 1020 is coupled to wand assembly 1040 by connection line 1035. Fan 926 works with suction system 1050 to pull fluid, solids and air from wand assembly 1040 through discharge line 1030 to the connected drain or vehicle mounted storage tank.

While the disclosure has been illustrated and described in detail in the drawings and foregoing description, the same is to be considered as illustrative and not restrictive in character, it being understood that only the preferred embodiments have been shown and described and that all changes and modifications that come within the spirit of the disclosure are desired to be protected.

1. A surface cleaning system comprising:
an external tank including a housing defining an internal area, wherein a tank vacuum motor is positioned within said internal area;
a wand assembly including a handle portion, a wand head, and a wand vacuum motor positioned between said handle portion and said wand head, wherein said wand vacuum motor is connected to a suction tube that extends from said vacuum motor to a position adjacent said wand head; and
a connection line extending between said external tank and said wand assembly to fluidly connect said external tank to said wand assembly;
wherein suction is provided to said suction tube by both said tank vacuum motor and said wand vacuum motor.

2. The surface cleaning system of claim 1, further comprising:
a liquid supply line in fluid connection with said handle portion of said wand assembly and wherein a liquid may be pumped through said liquid supply line to said wand head; and
a solution reservoir including a solution tank for holding a cleaning solution, wherein said solution reservoir is positioned adjacent to said liquid supply line at said handle portion of said wand assembly;
wherein said cleaning solution is introduced into said liquid in said liquid supply line.

3. The surface cleaning system of claim 2,
wherein said solution tank includes an entrance portion and an exit portion;
wherein said liquid supply line has a larger diameter at said entrance portion of said solution tank than at said exit portion of said solution tank; and,
wherein solution held in said solution tank is mixed with liquid from said liquid supply line at said exit portion of said solution tank due to the Venturi effect.

4. A surface cleaning system comprising:
a wand assembly including
a handle portion;
a wand head including a wand head cavity;
a wand vacuum motor positioned between said handle portion and said wand head, wherein said wand vacuum motor is adapted to provide suction to a suction tube extending from said vacuum motor to a position adjacent said wand head and to said wand head cavity; and
a valve positioned within said wand head, wherein said valve is movable between a first position that only allows suction to said suction tube and a second position that only allows suction to said wand head cavity;
wherein when said valve is in said first position, the surface cleaning system is adapted for use with dry cleaning and when said valve is in said second position, the surface cleaning system is adapted for use with steam cleaning.

5. A surface cleaning system comprising:
a liquid supply line attached to a liquid source that provides a liquid to said liquid supply line;
a wand assembly including a handle portion and a wand head in fluid connection with said liquid supply line;
a return tube in fluid connection with said liquid supply line; and
a valve including a first position, a second position, and a third position;
wherein when said valve is in said first position, said valve blocks said liquid supply line so that said liquid is diverted to said return tube;
wherein when said valve is in said second position, said valve allows a portion of said liquid to proceed through said liquid supply line while a portion of said liquid is diverted to said return tube; and
wherein when said valve is in said third position, said valve blocks said return tube so that said liquid continues through said liquid supply line.
